(19) United States
(12) Patent Application Publication
(10) Pub. No.: US 2007/0299535 A1
(43) Pub. Date: Dec. 27, 2007

(54) COATED ENOSSAL IMPLANT

(75) Inventor: Stefan Ihde, Uetliburg (CH)

Correspondence Address: HUSCH & EPPENBERGER, LLC, 190 CARONDELET PLAZA, SUITE 600, ST. LOUIS, MO, 63105-3441, US

(73) Assignee: Biomed Est., Vaduz (DE)

(21) Appl. No.: 11/768,398

(22) Filed: Jun. 26, 2007

(30) Foreign Application Priority Data
Nov. 29, 2006 (DE) DE 202006018188.1
Jun. 26, 2006 (DE) DE202006010202.7

(51) Int. Cl. A61C 8/00; A61F 2/28

(52) U.S. Cl. 623/23.6; 424/426; 433/201.1; 606/53

(57) ABSTRACT

The invention is an enossal implant with a coating that accelerates the healing-in process and counteracts demineralization of the bone when the cavities that are necessary for insertion of the implant are made. The invention elevates the osmotic pressure in the immediate vicinity of a freshly inserted implant so as to compensate quickly for the loss of salts and minerals of the chemical compounds and minerals contained in the bone and the bone fluid when the cavity is made. The surface of the implant may be, for example, coated with a dry crust of soluble NaCl crystals, which dissolves gradually after the insertion.

### CROSS-REFERENCE TO RELATED APPLICATIONS

This application claims priority to German application DE 20 2006 018 188.1 filed Nov. 29, 2006 and DE 20 2006 010 202.7 filed Jun. 26, 2006.

### BACKGROUND OF THE INVENTION

1. Field of the Invention

The present invention concerns an improved coated enossal implant which accelerates the healing-in process, and which is inserted into a cavity made in the bone.

2. Related Art

Maintaining the stability of enossal implants with respect to the bones into which they are placed is often a clinical problem. Mobility of implants is often observed both in orthopedic surgery and in dental and maxillofacial implantology. A certain portion of that mobility is due to infection. However, most of the mobility is caused by overloading the peri-implant bone. For instance, it is the most highly stressed screws, or the screws positioned in the least mineralized regions, such as in the tension or flexion regions of the bone, that become mobile in the case of fractured osteotomy plates.

The measures that have been known to limit or prevent these undesired processes amount to promoting new bone formation in the bony surgical region. Thus it has been suggested, among other things, to accelerate and stimulate the formation of new bony tissue by coating the implant surface with substances that promote bone growth.

Such procedures, and recommendations for coating of implants, are, for instance, known from DE 600 19 752 T2, DE 196 30 034 A1 and DE 196 28 464 A1. The measures known so far for coating implants relate predominantly to improved preparation of substrates for bone development, such as tricalcium phosphate, hydroxylapatite, and all sorts of calcium and phosphorus compounds. Measures for improved blood supply to the bone were also recommended to accelerate and stimulate formation of new bone tissue. Finally, increased provision of growth hormones and peptides of all types, which accelerate bone development, have been recommended.

None of those efforts has yet resulted in an actual useful and good clinical result, and there has been no overwhelming success in clinical practice, as it takes many weeks to months before the newly formed bone truly mineralizes and becomes capable of bearing a load. The implant mobility mentioned occurs much sooner, though.

To install an implant, an “implant bed” is produced in the jawbone with suitable drilling and grinding tools; a cooling liquid is used in the work done for that, which liquid carries out the fragments from the drilling and grinding, and it also cools the tool being used and the bone in the vicinity of the cavity or implant bed.

Physiologically compatible isotonic sodium chloride solutions are used for cooling in the current state of the art. They are commonly also known as infusion solutions, and were developed for infusions into veins and arteries. These infusion solutions are always available and can be obtained economically. The concept of “physiologically” compatible “isotonic” sodium chloride solution means that the solution in question is physiological, or isotonic, with respect to the blood of a patient, and generally has a sodium chloride concentration of 0.8% because that is the NaCl concentration in the blood. Thus a corresponding physiologically compatible sodium chloride solution is able to replace the blood volume in the blood vessels of a patient relatively well, within certain limits.

Aside from the physiologically compatible isotonic sodium chloride solutions, glucose solutions or Ringer's lactate are also used as the cooling liquid.

The common feature of the liquids named above that are used as cooling liquid is that the physiological concentration of ions or salts was selected with respect to the blood liquid of a patient. However, a “physiologically” compatible solution with a NaCl concentration of 0.8% or 0.9% has been discovered not to be physiological for the bones. The concentration prevailing in the bone liquid of ions and salts of sodium chloride, calcium, and phosphorus is higher than in the blood. Now if a physiologically compatible isotonic sodium chloride solution with a NaCl concentration of 0.8% is used as the cooling liquid in the drilling and grinding work that must be done, that results in leaching out or demineralization of the bone, which is disadvantageous for the healing-in process because the salts and other compounds, which are at higher concentrations in the bone, are dissolved out of the bone to compensate for the concentration in the cooling liquid. The region about the cavity thus loses its higher concentration of salts and other compounds, and in addition, the bones are weakened.

Implants are also known which are packaged by the manufacturer in a primary package filled with sodium chloride solution, and which are shipped wet in that form to the practice or hospital. The solution used is, again, a physiological sodium chloride solution with a NaCl concentration of 0.9%. Because of that NaCl concentration, this embodiment is also unable to solve the problems mentioned above, or to influence the solution of those problems in an advantageous manner. This form of packaging and the implant, wetted with the sodium chloride solution, which is removed from the packaging immediately before use, do not offer an advantage for bone healing.

### SUMMARY OF THE INVENTION

The invention concerns an enossal implant with a coating that accelerates the healing-in process and counteracts demineralization of the bone when the cavities that are necessary for insertion of the implant are made.

The invention elevates the osmotic pressure in the immediate vicinity of a freshly inserted implant so as to compensate quickly for the loss of salts and minerals of the chemical compounds and minerals contained in the bone and the bone fluid when the cavity is made.

According to the invention, the surface of the implant is, for example, coated with a dry crust of soluble NaCl crystals, which dissolves gradually after the insertion. In the bone, the high salt concentration of the coating results in a substantial inflow of interstitial liquid present in the bone to the implant, while the salt ions diffuse from the coating into the bone and compensate for the loss of minerals. At the same time, the implant is stabilized, and the elevated salt concentration prevents early colonization of bacteria.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is an enossal implant with a coating.

### DETAILED DESCRIPTION OF THE PREFERRED EMBODIMENTS

The following description of the exemplary embodiment(s) is in no way intended to limit the invention, its application, or uses.

The invention is directed towards a surface coating for implants, the substances of which will promote the regular osmotic relationships of the bone in the vicinity of the inserted implant, and compensate for the reduced amount or pressure of salts in the bony tissue due to the bleeding and to leaching from the bones because of the drilling and grinding processes.

In the implantology field for substances that can most quickly reproduce the regular osmotic conditions of the bone in the vicinity of the inserted implant and compensate for the reduced amount of pressure of salts in the bone tissue due to the drilling and grinding processes and to bleeding, it was surprisingly discovered that even a thin coating of the implant surface with sodium chloride produces such a local effect. The same is also true for a soluble coating of a calcium phosphate, CaSO₄ and other compounds present in the bone at high concentrations. Those substances show action similar to that of sodium chloride.

A local elevation of the concentration of the substances applied to the implant surface over the level found in blood, and their rapid solubility promote the quick reproduction of the regular osmotic relationships of the bone in the vicinity of the inserted implant. The substances of the coating advantageously do not adhere or have been applied solidly to the implant surface, as is the case for e.g. hydroxyapatite coatings; rather, they are advantageously able to diffuse easily into the adjacent osteonal systems of the bone after insertion.

It is also insufficient for the concentration of the substances in the coating, such as the NaCl concentration, to be at the usual physiologically compatible concentration of 0.8%, or 0.9% as is generally known for sodium chloride solutions. The concentration of the substances must be higher than their concentration in bones and in the bone liquid. Thus, for example, a coating with a higher NaCl concentration results in intraossal attainment of a substantial inflow of interstitial liquid present in the bones to the implant, while the salt ions of the coating diffuse away into the bone.

In this way, it is possible to reproduce the regular osmotic relations in the immediate vicinity of the implant immediately after the implantation in the bone. The implant is stabilized at the same time. An elevated salt concentration also inhibits early colonization with bacteria which are themselves ubiquitous.

One embodiment of the invention is distinguished by the sodium chloride concentration in the coating solution being higher than 0.9%. The extent to which the aforementioned concentration exceeds the level of 0.9% is preferably determined according to the invention solely by the salt concentration expected or previously found in the bone. It is advantageous to select the sodium chloride concentration so that it essentially corresponds to at least the plasma-isotonic or isoosmotic concentration with respect to the corresponding bone liquid. Therefore, in one embodiment preferred that the sodium chloride concentration in the coating solution is more than 0.95%, and preferably more than 1.0%.

In another embodiment, the concentration of potassium chloride corresponds essentially to at least the concentration of potassium chloride in the bone liquid in the bone in which the cavity is to be made.

According to a further feature of the invention, the concentration of potassium chloride may correspond substantially to at least the concentration of potassium chloride in the bone being treated or in the bone liquid in the vicinity of the cavity.

It may likewise be advantageous to combine the substances in the coating with antibiotics to combat any local infection or to be able to ward it off prophylactically. Individual representatives of the substances recommended according to the invention, such as calcium sulfate and calcium phosphate, are themselves antibiotically active. It may therefore be sufficient even to combine more of those substances named in the coating.

The coating according to the invention can, for example, be prepared by immersing an enossal implant, which may if possible have a roughened surface, in a sodium chloride solution at the desired concentration at the end of the cleaning process, and then carefully letting it dry. After the drying phase is completed, a thin, crust-like coating of sodium chloride remains on the surface of the implant. During and after insertion of the implant, this crust-like layer dissolves in the bone liquid and in the blood that is present locally. In this way, a site of higher salt concentration is produced in the bone, which reproduces the regular osmotic conditions of the bone in the vicinity of the inserted implant. It compensates for the reduction in the partial pressure of salts in the bone tissue that has been reduced by the leaching of the bone in the drilling and grinding process and by bleeding.

It was shown experimentally that even a physiological sodium chloride solution dried onto the implant surface has a quite different effect on the surroundings of the implant in the jawbone than an implant that had been removed from a prior art wet or liquid, physiological sodium chloride solution immediately before insertion. The dried-on salt molecules dissolve gradually, only after insertion of the implant. That results, advantageously, in a significantly higher local salt concentration in the vicinity of the inserted implant, which increases the amount of pressure of salt concentration and re-establishes the regular osmotic conditions in the shortest time.

It was established by resonance vibrations that implants coated according to the invention become fixed in the bone more rapidly than uncoated implants.

An advantage of the relatively simple solution according to the invention is principally that the recommended means act only on the osmotic relations. No medications are required for the rapid re-establishment of regular osmotic conditions in the vicinity of the inserted implant immediately after insertion. The active substances used, NaCl or the salts of other chemical compounds physiologically present in the bone or in the bone liquid, are identical with those of the body and so can be degraded without problem. Neither can any actual overdosing occur, because the inflow of liquid to the coated implant always causes a reduction (adaptation) of the salt concentration in the coating.

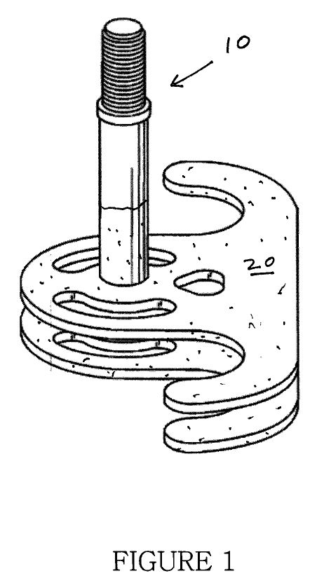

FIG. 1 shows an enossal implant 10 whose base and lower shaft have been coated 20 according to the process of the invention before implantation.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A coated implant comprising:
a bone implant; the implant surface having a coating of chemical compounds that are physiologically present in the bone and/or the bone liquid and which dissolve after said implant is implanted.

2. The coated implant according to claim 1, wherein the coating comprises salts of a calcium compound.

3. The coated implant according to claim 1, wherein the coating comprises salts of a calcium phosphate or calcium sulfate compound.

4. The coated implant according to claim 1, wherein the coating comprises salts of a potassium compound.

5. The coated implant according to claim 1, wherein said chemical is a salt concentration said salt concentration in said coating being greater than a salt concentration in blood.

6. The coated implant according to claim 5, wherein the salt concentration in the coating is equivalent at least to the salt content in the bone being treated or the bone liquid.

7. The coated implant according to claim 5, wherein the salt concentration in the coating is greater than the physiological concentration of the particular salt in the bone or the bone liquid.

8. The coated implant according to claim 5, wherein the salt concentration is greater than 0.8%.

9. The coated implant according to claim 5, wherein the NaCl concentration in the coating solution is greater than 0.9%.

10. The coated implant according to claim 5, wherein the NaCl concentration in the coating solution is greater than 0.95%.

11. The coated implant according to claim 5, wherein the NaCl concentration is greater than 1%.

12. The coated implant according to claim 1, wherein the coating is produced in the process of final cleaning of the implant, and that the implant is immersed in an appropriately prepared salt solution in connection with the final cleaning and the portions of the salt solution adhering to the implant surface are finally dried.

13. The coated implant according to claim 12, wherein the NaCl concentration of the washing solution before the drying of the implant to apply the coating is at least 0.9%.

14. Coated implant according to claim 12, wherein the NaCl concentration of the washing solution before the drying of the implant to apply the coating is from 1.0% to 20%.

15. The coated implant according to claim 1, wherein said implant is packaged in a primary package filled with a liquid NaCl solution and that the NaCl concentration in the solution is greater than 0.9%.

16. The coated implant according to claim 1, wherein said coating is a dry crust.

17. The coated implant according to claim 1, further comprising an NaCl solution of a primary packaging that is a gel.

18. The coated implant according to claim 1, wherein the active substances of the coating are combined with substances having antibiotic activity.

19. The coating according to claim 1, wherein the thickness of the coating is less than 2 μm.

20. The coating according to claim 1, wherein the proportion of the coated enossal implant surface is less than 100%.

21. The coated implant of claim 1 wherein said bone implant is a dental implant.
